(19) United States
(12) Patent Application Publication
(10) Pub. No.: US 2006/0057680 A1
(43) Pub. Date: Mar. 16, 2006

(54) Mutant interleukin-15 polypeptides

(76) Inventors: Xin Xiao Zheng, Wellesley, MA (US); Terry B. Strom, Brookline, MA (US); Thomas Moll, San Diego, CA (US)

Correspondence Address: FISH & RICHARDSON PC, P.O. BOX 1022, MINNEAPOLIS, MN, 55440-1022, US

(21) Appl. No.: 11/201,585

(22) Filed: Aug. 11, 2005

Related U.S. Application Data
(60) Provisional application No. 60/600,478, filed on Aug. 11, 2004.
(60) Provisional application No. 60/601,042, filed on Aug. 11, 2004.

(51) Int. Cl. C07K 14/54; C07H 21/04; C12P 21/04

(52) U.S. Cl. 435/69.52; 435/320.1; 435/325; 530/351; 536/23.5

(57) ABSTRACT

Mutant IL-15 polypeptides and compositions including the polypeptides are described herein. In various embodiments, a mutant IL-15 polypeptide is joined to a heterologous polypeptide. Also described herein are uses of the mutant IL-15 polypeptides, e.g., in suppressing immune responses.

### RELATED APPLICATIONS

This application claims the benefit of U.S. provisional applications 60/600,478 and 60/601,042, both filed Aug. 11, 2004. For the purpose of any U.S. patent that may issue from the present application, the entire contents of the prior provisional applications are hereby incorporated by reference.

### FUNDING

Some of the work described herein was supported by a grant from the National Institutes of Health. The United States government may therefore have certain rights in the invention.

### TECHNICAL FIELD

This invention relates to mutant interleukin-15 (IL-15) polypeptides, and more particularly to clinically useful polypeptides that include a mutant IL-15 polypeptide and an Fc region of an immunoglobulin.

### BACKGROUND

IL-15 was purified based on its ability to support the proliferation of a mouse T cell line (Grabstein et al., Science 264:965, 1994). When the gene encoding IL-15 was isolated and sequenced, it was found to predict a mature protein containing 114 amino acid residues, formed by cleavage of a precursor having 162 amino acid residues (see Krause et al., Cytokine 8:667-674, for the genomic sequence).

Human IL-15 is highly expressed in the placenta, monocytes of peripheral blood, and skeletal muscle. It is weakly expressed in heart, lung, liver, kidney, and several other tissues. IL-15 is believed to support the differentiation and proliferation of T cells and B cells; to activate natural killer (NK) cells; and to activate cytotoxic T lymphocytes (CTLs) and lymphokine-activated killer (LAK) cells. In the context of an immune response, IL-15 stimulates the proliferation and differentiation of lymphocytes.

IL-15 exerts its influence by binding to a cell surface receptor that consists of three distinct subunits: an IL-2Rβ subunit, an IL-2Rγ subunit, and a unique IL-15Rα subunit. IL-15 binding is thought to stimulate activation of two receptor-associated kinases, Jak1 and Jak3 (Caliguiri, Blood 97:14, 2001). Jak1 and Jak3 activation results in phosphorylation of two signal transducer and activator of transcription (STAT) proteins, STAT3 and STAT5 (Caliguiri, Blood 97:14, 2001).

### SUMMARY

We have previously generally disclosed mutants of an IL-15 polypeptide and chimeric polypeptides that include a mutant IL-15 and a heterologous polypeptide (see, e.g., U.S. Pat. No. 6,451,308). Here, we describe more specific IL-15 mutants and variants thereof, including variants that contain a leader sequence, whether of naturally occurring IL-15 or another protein (e.g., a CD5 leader sequence) and variants in which the mutant IL-15 polypeptide is joined to one or more heterologous polypeptides.

While the IL-15 mutants are described further below, we note here that the present invention encompasses mutant polypeptides that include the polypeptide sequence of a naturally occurring IL-15 having (a) a mutation (e.g., a deletion mutation) of one or more of the first 48 amino acid residues of the precursor protein and (b) a mutation (e.g., a substitution mutation) of one or both of the glutamine (Q) residues in the C-terminal half of the polypeptide. Such IL-15 mutants can be part of a fusion protein, including those that contain a leader sequence and/or a heterologous (i.e., non-IL-15) sequence, such as the Fc region of an IgG molecule. As with IL-15, the leader sequence or heterologous sequence can be mutant with respect to their wild-type counterparts. Mutants of the Fc region, as described herein, are another aspect of the invention. These Fc mutants can be fused or otherwise joined to other polypeptides (e.g., IL-15), regardless of whether the other polypeptide is mutant or wild-type (e.g., the Fc mutants described herein can be fused to a wild-type IL-15 or any other growth factor (e.g., any other interleukin).

Where the IL-15 is mutant, the glutamine residue(s) within the C-terminal of IL-15 ( e.g., the glutamine residues at positions 101 and 108 of SEQ ID NO:1) can be replaced with aspartic acid (D) or with any other naturally or non-naturally occurring amino acid residue. Regardless of the substituted residue, the change can be considered “conservative” or “non-conservative” (non-limiting examples of which are provided below). The naturally occurring IL-15 that is mutated can be a human IL-15, and the mutation can produce the sequence represented by SEQ ID NO:2. Any of the mutant IL-15 polypeptides described herein can be joined to a leader sequence (e.g., a CD5 leader sequence (SEQ ID NO:3)). The leader sequence can serve as a signal sequence that directs the mutant IL-15 polypeptide through a cell in which it is expressed (e.g., a cell of a COS cell line or a Chinese hamster ovary (CHO) cell line) and to the extracellular space. The leader sequence can include one or more mutations (e.g., a substitution or deletion mutation of one, two, three, four, five, six, or seven amino acid residues) so long as it retains the ability to direct protein secretion. In one embodiment, the mutant IL-15 comprises SEQ ID NO:4. In one embodiment, the mutant IL-15 polypeptide consists of SEQ ID NO:4.

As noted, any one of the mutant IL-15 polypeptides can be joined to one or more heterologous polypeptides. The non-IL-15 portion of these chimeras may increase the circulating half-life of the mutant IL-15 polypeptide, serve as a label or tag (e.g., an antigenic tag or epitope tag), or confer some other desirable quality on the mutant IL-15. We use the term “circulating half-life” in the conventional sense to refer to the period of time that elapses before a given amount of a substance that is present in the circulatory system of a living animal (e.g., a human patient) is reduced by one half.

The heterologous polypeptide can be, for example, serum albumin or the Fc region of an immunoglobulin. These polypeptides can have the same amino acid sequences they have in nature or they can contain at least one mutation (e.g., up to about 10% of the sequence can be mutated). The mutation(s) can be conservative or non-conservative. In various embodiments, a mutation is a substitution mutation. For example, the Fc region can include a substitution mutation of the first N-terminal cysteine residue (shown at position 5 of SEQ ID NO:5). That cysteine residue can be replaced, for example, with an alanine (A) residue. Alternatively, or in addition, the initial amino acid residue of the “hinge” within the Fc region can be mutated. For example, the initial glutamic acid residue (E) of SEQ ID NO:5 can be changed to an aspartic acid (D) residue.

Additional mutations can render the Fc region non-lytic (see below), and Fc polypeptides that include these mutations are also within the scope of the present invention. Additional mutations (i.e., mutations that do not affect lytic function) can also be made so long as the desired functional attributes of the polypeptide are retained. For example, when a polypeptide is joined to a mutant IL-15 for the purpose of increasing the IL-15's circulating half-life, that polypeptide can differ from a corresponding wild-type sequence so long as it retains the ability to prolong half-life. For example, a mutant serum albumin that contains either more or less amino acid residues than wild-type serum albumin (i.e., addition or deletion mutants, respectively) can be used, as can a polypeptide in which one or more amino acid residues have been substituted (e.g., about 1-5, 1-10, 10-20, 15-25, or 25-50% of the amino acid residues). As with IL-15, the substitution(s) can be considered conservative or non-conservative. We may refer to molecules containing both IL-15 and a non-IL-15 polypeptide as chimeric polypeptides (e.g., a polypeptide that includes a mutant IL-15 joined to an Fc region may be referred to as a mutant IL-15/Fc chimera).

In the paragraphs above (and further below), we describe mutant IL-15 polypeptides that can be, but are not necessarily, joined to wild type or mutant Fc regions. As noted, as we have made unique Fc regions, and those Fc regions and polypeptides containing them are also within the scope of the present invention. The mutant Fc regions can be expressed alone, joined to any mutant IL-15 (including those described herein, as noted above), or any other polypeptide (e.g., a biologically active polypeptide such as a wild type IL-15, another interleukin (e.g., IL-1, IL-2, IL-7, IL-10, or IL-21), or another cytokine (e.g., brain-derived neurotrophic factor (BDNF), epidermal growth factor (EGF), a fibroblast growth factor (FGF), glial growth factor (GGF), or nerve growth factor (NGF)). Regardless of the precise configuration or sequence, the Fc region can be that of, or can be derived from (i.e., can be a mutant form of), the Fc region of any immunoglobulin. For example, when a naturally occurring Fc region is joined to a mutant IL-15, the Fc region can be that of an immunoglobulin of the A, D, E, G or M class (i.e., an IgA, IgD, IgE, IgG, or IgM). Each of these types of immunoglobulins can be obtained from a human subject. In one embodiment, the Fc region is an Fc region of human IgG1. Similarly, when a mutant Fc region (as described herein) is used, the mutant Fc region can be a mutant of an IgA, IgD, IgE, IgG or IgM. In one embodiment, the mutant Fc region is a mutant human IgG1 Fc region.

When joined by peptide bonds, an IL-15/Fc chimera can have the sequence of SEQ ID NO:7. The Fc region, whether expressed alone or as part of a chimeric polypeptide (e.g., a mutant IL-15/Fc chimera), can include a leader sequence, such as the CD5 leader sequence.

The IL-15 molecules (e.g., the mutant IL-15 molecules described herein, alone or fused to a heterologous polypeptide) can be chemically modified by conjugation to a water-soluble polymer such as polyethylene glycol (PEG), e.g., to increase stability or circulating half-life.

The polypeptides of the invention can be, but are not required to be, substantially free of heterologous biological agents (as the polypeptides and nucleic acids of the invention are mutants or chimeras, we do not expect them to occur in nature; isolation or purification is therefore not necessary to distinguish the compositions of the present invention from compositions found in nature). Where a polypeptide is substantially pure, it can be at least or about 50% (e.g., at least about 55, 60, 65, 70, 75, 80, 85, 90, 95, 97, or 99% pure). As noted, purity can be assessed with respect to heterologous biological agents, which include non-IL-15 polypeptides, other proteins, and cellular material such as lipids and nucleic acids.

The mutant IL-15/Fc polypeptides described herein can be dimerized, and such dimers are within the scope of the present invention. The dimer can consist of two identical polypeptides (e.g., two copies of the polypeptide represented by SEQ ID NO:7) or two non-identical polypeptides (one of which can be the polypeptide represented by SEQ ID NO:7). Regardless of the precise polypeptides used, the C-termini and N-termini can be aligned or roughly aligned. For example, where each of the polypeptides includes an Fc region at the N-terminus, the dimer can include molecular bonds between the two Fc regions (e.g., disulfide bonds between one or more of the cysteine residues within one Fc region and the other).

In another aspect, the invention features nucleic acid molecules that encode any of the polypeptides described herein (e.g., the mutant IL-15 polypeptide described herein, the Fc region described herein, and chimeric polypeptides containing them). The sequences of the nucleic acid molecules can vary due to the degenerate nature of the genetic code.

The polypeptide-encoding nucleic acids can be contained within expression vectors (e.g., plasmid or viral vectors), which are also within the scope of the present invention. Moreover, the nucleic acid molecules and vectors of the present invention can be contained within cells (e.g., CHO cells), and such genetically modified cells are also within the scope of the present invention. The invention also features methods of making the polypeptides described herein by providing host cells that express the encoded protein (e.g., a mutant IL-15 polypeptide as described herein). For example, the cells can be expanded in tissue culture (e.g., a liquid culture) under conditions that permit protein expression. The expression vector can include sequences that may facilitate expression or direct secretion of the expressed protein. For example, the vector can include a promoter or enhancer, a sequence encoding a leader or signal sequence (e.g., an IL-15 leader or that of another interleukin (e.g., IL-1 (e.g., IL-1α or IL-1β) IL-2 (see Bamford et al., J. Immunol. 160:4418, 1998) IL-4, or IL-10), a CD5, CTLA4, or TNF leader), and a polyadenylation signal. The leader sequence may be as found in nature or may be truncated or otherwise mutated; what is required is that enough of the wild-type sequence is retained to allow the leader to function (e.g., to allow sufficient secretion of the mature protein to which it was attached within the cell). The vector can also include a selectable marker, such as a sequence encoding a protein that confers antibiotic resistance (e.g., resistance to G418). The expressed protein can be purified from host cells or from culture supernatants using purification methods known in the art (for example, protein can be purified from culture supernatants by protein A Sepharose™ affinity chromatography followed by dialysis against PBS and, optionally, filter sterilization). Due to their length, we expect the polypeptides described herein to be obtained by recombinant methods, but chemical synthesis is also possible.

The nucleic acid molecules may be contained within a vector that is capable of directing expression of a mutant IL-15 polypeptide in, for example, a cell that has been transduced (e.g., transfected) with the vector. These vectors may be viral vectors, such as retroviral, adenoviral, or adenoviral-associated vectors, as well as plasmids or cosmids. More specifically, the vector can be a modified herpes virus, simian virus 40 (SV40), papilloma virus, or a modified vaccinia Ankara virus.

Suitable vectors include T7-based vectors for use in bacteria (see, e.g., Rosenberg et al., Gene 56:125, 1987), the pMSXND expression vector for use in mammalian cells (Lee and Nathans, J. Biol. Chem. 263:3521, 1988), and baculovirus-derived vectors (for example, the expression vector pBacPAK9 from Clontech, Palo Alto, Calif., USA) for use in insect cells. While additional promoters are described elsewhere, we note that a T7 promoter can be used when the host cells are bacterial, and a polyhedron promoter can be used in insect cells.

Mammalian expression vectors typically include nontranscribed regulatory elements such as an origin of replication, a promoter sequence, an enhancer linked to the structural gene, other 5′ or 3′ flanking nontranscribed sequences (e.g., ribosome binding sites, a polyadenylation site, splice donor and acceptor sites, and transcriptional termination sequences). Regulatory sequences derived from Polyoma, Adenovirus 2, Simian Virus 40 (SV40), and human cytomegalovirus are frequently used for recombinant expression in mammalian cells. For example, SV40 origin, early and late promoter, enhancer, splice, and polyadenylation sites may be used to provide the other genetic elements required for expression of an IL-15 mutant DNA sequence in a mammalian host cell. Cytomegalovirus or metallothionein promoters are also frequently used in mammalian cells.

Cells (e.g., eukaryotic cells) that contain and express a nucleic acid molecule encoding any of the mutant IL-15 polypeptides described herein are also features of the invention, and they can be used in methods of making the mutant IL-15-containing polypeptides described herein or administered to patients receiving a transplant (e.g., a heart transplant, lung transplant, or heart-lung transplant) or otherwise in need of modulating the IL-15-mediated part of an immune response.

Examples of suitable mammalian host cell lines for production of mutant IL-15 polypeptides include: CHO cells; COS cell lines derived from monkey kidney, (e.g., COS-7 cells, ATCC number CRL 1651); L cells; C127 cells; 3T3 cells (ATCC number CCL 163); HeLa cells (ATCC number CCL 2); and BHK (ATCC number CRL 10) cell lines.

In addition to compositions such as those described above, the invention further features compositions and methods of improving a patient's status or prognosis following transplantation (e.g., graft function or survival) or in the event of an autoimmune disease, vascular injury, or other event associated with an IL-15-dependent immune response by administering one or more types of IL-15 or IL-15R antagonists and an agent that inhibits CD40L (also known as CD154). The agent that inhibits CD40L can be, e.g., an anti-CD154 antibody or an antigen-binding fragment thereof; a soluble monomeric CD40L, an inhibitory nucleic acid such as an antisense RNA molecule or siRNA that specifically binds a nucleic acid sequence encoding CD40L or a small molecule (e.g., a small organic molecule). Accordingly, pharmaceutical compositions that include an IL-15 or IL-15R antagonist and an agent that inhibits CD40L are within the scope of the present invention, as are kits that include these compositions, in the same or separate containers, and methods of using them. Other combination therapies within the invention include administration of a combination of one or more antagonists of IL-15 or IL-15R. For example, one can administer a mutant IL-15 polypeptide as described herein and an antibody that binds IL-15 or an IL-15R and inhibits signal transduction. Such antibodies are known in the art and are available from the American Type Culture Collection (ATCC, Rockville, Md. (USA)).

In specific embodiments, the invention features methods of making the mutant IL-15 polypeptide, the mutant Fc region, or chimeric polypeptides containing either or both of these polypeptide sequences. The methods can be carried out by synthesizing the amino acid sequences or by recombinant methods. For example, one can make a polypeptide of the invention by providing a cell that expresses a nucleic acid molecule that encodes the desired polypeptide (e.g., SEQ ID NO:7); culturing the cell under conditions and for a time sufficient to allow expression of the polypeptide; and isolating the polypeptide from the cell. The polypeptide can be crudely or highly purified by methods known to one of ordinary skill in the art (by, e.g., chromatography, as noted above). For example, the polypeptide can be substantially free of heterologous biological agents.

The polypeptides (whether in a monomeric or dimeric form), nucleic acids, vectors, and genetically modified cells can be formulated for administration to a patient. Accordingly, the invention features pharmaceutically acceptable compositions comprising an amount (e.g., a therapeutically effective amount) of the mutant IL-15 polypeptide, the mutant Fc region, or chimeric polypeptides containing either or both of those polypeptides (i.e., either or both of a mutant IL-15 polypeptide and the mutant Fc region).

Other methods of the invention concern suppression of the immune response and treatment of immune-related diseases or disorders, particularly those caused by, or exacerbated by, activation of cells that express an IL-15 receptor complex. For example, the invention features methods of suppressing the immune response in a patient by administering to the patient a mutant IL-15 polypeptide described herein (or a chimeric polypeptide that includes such a polypeptide) or a nucleic acid molecule encoding the polypeptide, or recombinant cells (e.g., human cells) that secrete it. The amount will be sufficient to inhibit one or more of the cellular events that normally occur as a consequence of interaction between wild type IL-15 and the IL-15 receptor complex. The patient may be one who has been diagnosed as having, or who is predisposed to having, an autoimmune disease such as a rheumatic disease (e.g., rheumatoid arthritis, systemic lupus erythematosus, Sjögren's syndrome, scleroderma, mixed connective tissue disease, dermatomyositis, polymyositis, Reiter's syndrome, and Behcet's disease). The autoimmune disease can also be psoriasis, type I diabetes, or an autoimmune disease of the thyroid (e.g., Hashimoto's thyroiditis and Graves' Disease). Other patients amenable to treatment include those having an autoimmune disease of the central nervous system (e.g., multiple sclerosis, myasthenia gravis, or encephalomyelitis) or a variety of phemphigus (e.g., phemphigus vulgaris, phemphigus vegetans, phemphigus foliaceus, Senear-Usher syndrome, and Brazilian phemphigus). Other patients amenable to treatment include those infected with a human immunodeficiency virus (HIV (e.g., HIV type 1 or HIV type 2)).

In other embodiments, the invention features methods of treating a patient who has received, or who is scheduled to receive, a transplant of a biological tissue or a device that includes a biological tissue. The patient's immune system can be targeted, as described above, by administering a therapeutically effective amount of one or more of the types of mutant IL-15 polypeptides described herein. Alternatively, the patient can be treated by administering a nucleic acid molecule encoding the mutant IL-15 or a population of recombinant cells expressing the mutant IL-15 polypeptide (e.g., a population including human cells, at least some of which may be the patient's own cells). The biological tissue can be essentially any biological tissue, and it can be, or can include, cells, portions of organs, and/or whole organs (no specific anatomical structure is intended by the use of the term “tissue”). More specifically, the patient may have received, or be scheduled to receive, a biological tissue (a “transplant”) or device that includes islet cells (or other cell types from the endocrine system), cardiac, smooth, or skeletal myocytes, epithelial cells (or other cell types within skin), hepatocytes, osteocytes (or other cell types within bone or other connective tissue), neurons, glial cells, or tissue from the lung, vascular system, urinary system, or reproductive system.

Where a patient has experienced, or is at risk of experiencing, unwanted proliferation of cells that express an IL-15 receptor complex, they can be treated with a mutant IL-15, a chimeric polypeptide that includes a mutant IL-15 polypeptide, a nucleic acid that encodes mutant IL-15 or a chimeric polypeptide of which it is a part, or a cell (e.g., an autologous cell) that expresses the mutant or chimeric polypeptide. Although the methods are not limited to those in which cellular proliferation is inhibited by one mechanism or another, cellular proliferation may be inhibited by complement directed cytolysis or antibody dependent cellular cytotoxicity. For example, a mutant IL-15/Fc chimera can be used to treat a patient who has received, or who is expected to receive, a vascular injury. Such patients include those who have undergone, or who are scheduled to undergo, an angioplasty (e.g., a balloon angioplasty) or other procedure that results in restenosis (e.g., a coronary artery bypass graft or percutaneous mitral valvuloplasty (PMA), which may be initiated by the recurrence of acute rheumatic fever). Unwanted proliferation may also occur in any of the cell types that express the IL-15 receptor complex or cells to which IL-15 is presented. These cells include macrophages (e.g., mitogen-activated macrophages), natural killer (NK) cells, and T cells (CD4⁺ and CD8⁺). Thus, patients having cancers in which these cell types proliferate (e.g., a leukemia or lymphoma) are amenable to treatment. The methods of the present invention can be carried out in conjunction with other therapies (e.g.. chemotherapy or radiation therapy).

Preferably, the mutant IL-15 polypeptides of the present invention bind the IL-15 receptor complex with an affinity similar to wild type IL-15, but fail to activate signal transduction. Such mutants will compete effectively with wild-type IL-15 and block the events that normally occur in response to IL-15 signalling, such as cellular proliferation.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1A-1H are representations of amino acid sequences represented by SEQ ID Nos:1-8, respectively.

FIG. 2 is a representation of the sequence of a human mutant IL-15 fused to a human IgG1 Fc molecule. A leader sequence is also shown (represented by negative numbers and misaligned (SEQ ID NO:3). The mutant IL-15 sequence is numbered in the figure as residues 1-114. The sequence numbered in the figure as residues 115-346 is an Fc region including the hinge and segments C2 and C3. The fused mutant IL-15 sequence and the Fc region are represented by SEQ ID NO:7. Glycosylation sites are underlined and point mutations are highlighted with arrows.

### DETAILED DESCRIPTION

Mutant IL-15polypeptides and variants thereof: The mutant IL-15 polypeptides of the invention are polypeptides that differ from IL-15 polypeptides found in nature in two specific ways. First, the present mutants lack one or more of the first 48 amino acid residues (i.e., the N-terminal residues) of the IL-15 precursor protein and, second, include a substitution mutation of one or both of the glutamine (Q) residues in the C-terminal half of the polypeptide. With respect to the deletion, it may be of 1, 2, 3, 5, 10, 12, 15, 20, 25, 30, 35, 40, 45, or 48 of the 48 most N-terminal residues of the precursor protein. Where anything less than all 48 residues are deleted, the deletion can begin at the first residue and may or may not include contiguous amino acid residues. With respect to the substitution mutation(s), each glutamine residue can be replaced with one or more (e.g., 1, 2, 3, 5, 10, or more) amino acid residues. For example, the glutamine residue can be replaced with a single amino acid residue (i.e., the substitution mutation can be a point mutation), which may be aspartic acid (D) or any other naturally or non-naturally occurring amino acid residue. Where a glutamine residue is replaced with a glutamic acid (E), aspartic acid (D), or asparagine (N) residue, the substitution may be referred to as a “conservative” substitution. Where the substitution is made with a different naturally occurring amino acid residue (i.e., a residue other than E, D, or N), the substitution may be referred to as a “non-conservative” substitution. Synthetic or “unnatural” amino acid residues can also be used. For example, one or both glutamine residues can be replaced with squaramine, an analog of glutamic acid (Chan et al., J. Med. Chem. 38:4433, 1995).

The mature, naturally occurring IL-15 that is mutated can be that of any mammal or any other animal that expresses an IL-15. For example, the IL-15 can be that of a rodent (e.g., a mouse, hamster, guinea pig, or rat), a domesticated animal (e.g., a dog or cat), a wild animal (e.g., a rabbit or hare, fox, deer, or coyote), a farm animal (e.g., a horse, cow, buffalo, llama, pig, sheep, or goat), a non-human primate (e.g., a monkey, ape, gorilla, or chimpanzee), or a human being. The mutation can produce the sequence represented by SEQ ID NO:2.

Any one of the mutant IL-15 polypeptides described herein can be joined to one or more heterologous polypeptides, which may constitute all, or a part of, a naturally occurring protein. The non-IL-15 portion of the chimeric polypeptide may increase the circulating half-life of the mutant IL-15 polypeptide, serve as a label or tag (e.g., an antigenic tag or epitope tag), or confer some other desirable quality on the mutant IL-15. For example, the IL-15 mutant, or any variant thereof (including the chimeric polypeptides described further below), can be joined to an epitope tag such as c-myc or FLAG®. These sequences are fused to the N- or C-terminus of the expressed IL-15 polypeptides, making them more accessible for antibody detection. The original FLAG sequence (DYKDDDDK (SEQ ID NO:__) is recognized by two monoclonal antibodies, M1 and M2 (Hopp et al., BioTechnology 6:1204-1210, 1988; Prickett et al., BioTechniques 7:580-589, 1989). In addition, the FLAG sequence with an initiator methionine attached is recognized by the M2 antibody and a third antibody, M5 (Brizzard and Chubet in Current Protocols in Neuroscience, Crawley, J. N., et al., Eds, pp. 5.8.1-5.8.11 (John Wiley & Sons, New York, N.Y., 1999). As the last five amino acids of the FLAG sequence are the recognition site for the protease enterokinase, the FLAG epitope tag can be removed from another protein by digestion with this enzyme (see also Blanar et al., Science 256:1014, 1992; LeClair et al., Proc. Natl. Acad. Sci. USA 89:8145, 1992). A heart muscle kinase (HMK) recognition site can also be used to allow introduction of a radioactive label (e.g., ³²P) into the polypeptide (Blanar et al., Science 256:1014, 1992; LeClair et al., Proc. Natl. Acad. Sci. USA 89:8145, 1992). For example, the HMK sequence can be fused to a FLAG sequence as well as to a mutant IL-15 polypeptide or a chimera containing the mutant IL-15 polypeptide.

The heterologous polypeptide can also be serum albumin (e.g., human serum albumin) or a portion thereof sufficient to increase circulating half-life (e.g., one or more of the domains referred to as domains I, II, and III). The amino acid sequence of the serum albumin may be naturally occurring or contain substitutions (e.g., about 1-2, 1-5, 2-5, 1-10, 5-10, 10-20, 15-20, 15-25, 20-25, 25-30, or 25-50% of the amino acid residues can be replaced with conservative, non-conservative, or unnatural amino acid residues). Circulating half-life may be increased, regardless of the polypeptide sequence used, to any clinically beneficial extent (e.g., two-, three-, or four-fold or more).

Alternatively, the mutant IL-15 polypeptide can be joined to an Fc region (fragment crystilizable) of an immunoglobulin, which is the fragment obtained when an immunoglobulin (e.g., IgG) is digested with papain. The Fc region can be glycosylated and can carry out any of the effector functions normally carried out by the Fc region (e.g., binding complement or cell receptors), even when joined to the mutant IL-15 polypeptide. The Fc region also carries the antigenic determinants that distinguish one class of antibody from another, and the Fc region of any class can be joined to the mutant IL-15 polypeptides described herein. For example, the Fc region can be that of the A, D, E, G, or M class (i.e., an IgA, IgD, IgE, IgG, or IgM) or a subgroup thereof (e.g., IgG1, IgG2, IgG3, or IgG4). Each of these types of immunoglobulins can be obtained from a human subject.

When mutated, the sequence of the Fc region can differ significantly from that of its wild type counterpart, so long as it retains the ability to prolong the half-life of a polypeptide to which it is joined or confers some other desirable property on a chimeric polypeptide of which it is a part. The amino acid sequence of the Fc region may contain contiguous or non-contiguous deletions of one or more amino acid residues (e.g., deletions of 1-2, 2-3, 3-5, 5-10, 10-15, 15-30, 30-50, 50-100, or 100-200 residues). Alternatively, or in addition, the Fc region may contain one or more substitutions (e.g., 1-2, 2-3, 3-5, 5-10, 10-15, 15-30, 30-50, or 50-100 of the amino acid residues can be replaced with conservative, non-conservative, or unnatural amino acid residues). More specifically, the Fc region can include a substitution mutation of the first N-terminal cysteine residue (shown for an IgG at position 5 of SEQ ID NO:5). That cysteine residue can be replaced, for example, with an alanine (A) residue. Alternatively, or in addition, the initial amino acid residue of the “hinge” within the Fc region can be mutated. For example, the initial glutamic acid residue (E) of an IgG an be changed to an aspartic acid (D) residue.

While the Fc region may be lytic (i.e., able to bind complement or to lyse cells via another mechanism, such as antibody-dependent complement lysis (ADCC); see U.S. Pat. No. 6,410,008), mutations can be introduced that render the Fc region non-lytic. Such mutants would inhibit complement fixation and Fc receptor binding. For example, the mutant Fc can lack a high affinity Fc receptor binding site and a C′1q binding site. As the high affinity Fc receptor binding site includes the leucine residue at position 235 of IgG Fc, Fc receptor binding can be diminished by deleting or changing that amino acid residue. For example, substituting glutamic acid (E) for that leucine residue inhibits the ability of the Fc region to bind the high affinity Fc receptor. The C′1q binding site can be functionally destroyed by deleting or changing the glutamic acid residue at 318, the lysine residue at 320, and the lysine residue at 322 of IgG1. For example, substituting alanine residues for Glu 318, Lys 320, and Lys 322 renders IgG1 Fc unable to direct ADCC. The complement (C1q) and FcγR1 binding sites of a human Fcγ1 fragment can be mutated to produce a nonlytic form of a human Fc-related fusion protein. For further information regarding C1q, one can consult Duncan and Winter (Nature 332:738, 1988) and for additional information regarding FcγR1, Duncan et al. (Nature 332:563, 1988), which are hereby incorporated by reference in the present application.

The mutant Fc regions described herein (e.g., the polypeptides in which the first amino acid residue of the hinge region and the first N-terminal cysteine residue are substituted with, for example, aspartic acid and alanine, respectively) can be expressed alone, joined to any mutant IL-15 (including those described herein, as noted above), or any other polypeptide (e.g., a wild type IL-15, another interleukin (e.g., IL-1, IL-2, IL-3, IL-4, IL-5, IL-6, IL-7, IL-8, IL-9, IL-10, IL-11, IL-12, IL-13, IL-15, IL-16, IL-17, IL-18, or IL-21), or another cytokine or growth factor (e.g., a brain-derived neurotrophic factor (BDNF), an epidermal growth factor (EGF), a fibroblast growth factor (FGF), GM-CSF, G-CSF, an interferon (e.g., IFN-α, IFN-β, and IFN-γ), a tumor necrosis factor (e.g., TNF-α or TNF-β), a glial growth factor (GGF), or a nerve growth factor (NGF)). Regardless of the precise configuration or sequence, the Fc region can be that of, or can be derived from (i.e., can be a mutant form of), the Fc region of any immunoglobulin class or subclass. When joined by peptide bonds, an IL-15/Fc chimera can have the sequence of SEQ ID NO:7.

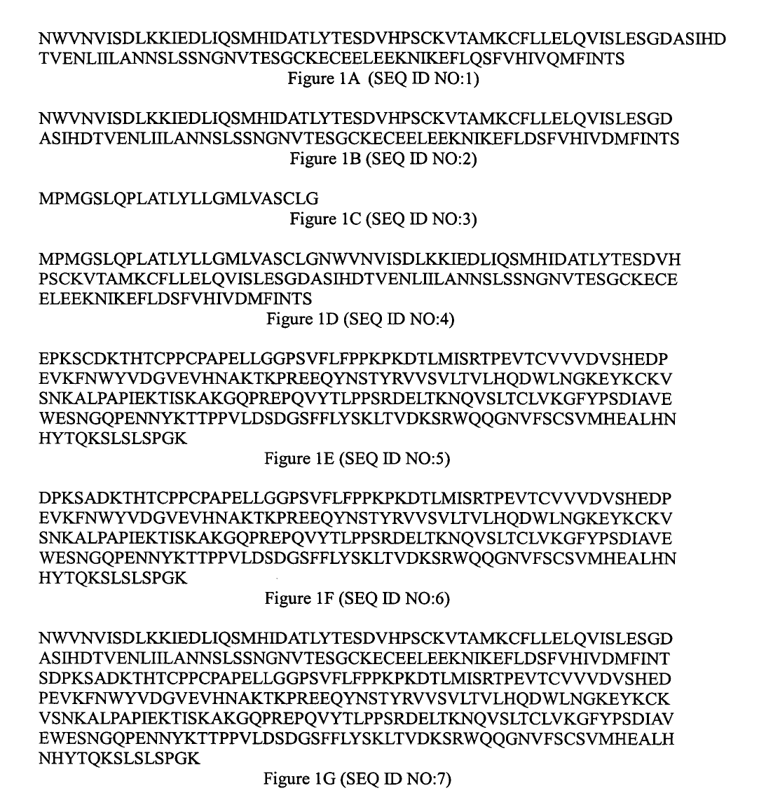

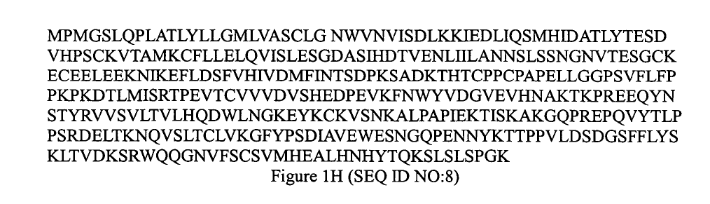

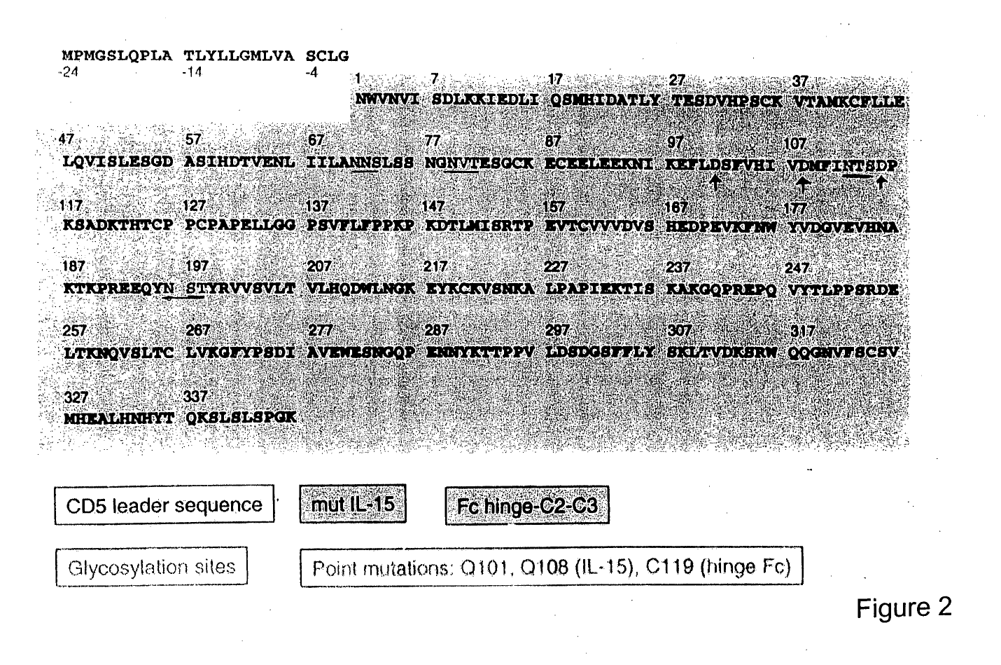

Any of the mutant IL-15 polypeptides described herein, whether expressed alone or as part of a chimeric polypeptide, can be joined to a leader sequence (e.g., a CD5 leader sequence (SEQ ID NO:3)). The leader sequence can serve as a signal sequence that directs the mutant IL-15 polypeptide through a cell in which it is expressed and to the extracellular space. The leader sequence can be about 15 to about 25 amino acid residues long and capable of targeting proteins to which it is attached to the endoplasmic reticulum. As an alternative to the CD5 leader sequence shown in FIG. 1, the leader sequence can be MRYMILGLLALAAVCSA (SEQ ID NO:9), a signal sequence derived from the adenovirus type 5, E3/19 K gene product (Persson et al., Proc. Natl. Acad. Sci. USA 77:6349-6353, 1980). Other suitable leader sequences are known in the art (see, e.g., van Heijne, J. Mol. Biol. 184:99-105, 1985).

With respect to the sequence of the mutant IL-15, in various embodiments, such polypeptides will be at least or about 65% (e.g., at least or about 63, 64, 65, 66, or 67%) identical to a wild type IL-15; at least or about 75% (e.g., at least or about 73, 74, 75, 76, or 77%) identical to a wild type IL-15; at least or about 85% (e.g., 83, 84, 85, 86, or 87%) identical to a wild type IL-15;, or at least or about 90% (e.g., 89, 90, 91, 92, 93, 94, 95, 96, 97, 98, or 99%) identical to a wild type IL-15. The mutant and wild type polypeptides compared can be of the same species. For example, the wild type IL-15 can be a human IL-15, and one can introduce mutations into the human sequence to produce a mutant IL-15. The wild type sequence may be referred to as the reference standard. Moreover, the referenced wild type sequence and the mutant to which it is compared can constitute a mature form of an IL-15 or a precursor that includes a signal peptide. More specifically, the wild type sequence and the mutant to which it is compared can constitute a form of IL-15 that includes the signal peptide MVLGTIDLCSCFSAGLPKTEA (SEQ ID NO: ______) followed by amino acid residues constituting a mature form of IL-15. The mutant IL-15 polypeptides can: (a) include a mutation at position 149 of SEQ ID NO:2, (b) exhibit at least 90% identity to a corresponding wild type IL-15, and (c) inhibit one or more of the activities mediated by wild type IL-15.

A wild type IL-15 polypeptide that is joined to (e.g., fused to) a heterologous polypeptide can also serve as a reference standard for a corresponding mutant protein. For example, a wild type IL-15 polypeptide fused to a wild type Fc region of an immunoglobulin can serve as the reference standard for a mutant IL-15 polypeptide fused to a mutant or wild type Fc region of an immunoglobulin. Such agents can exhibit the same certain degrees of identity to a corresponding reference standard as set forth above with respect to IL-15 alone. For example, where the agent includes a mutant IL-15 and an Fc region, the mutant IL-15 and Fc region can be at least or about 90% (e.g., 89, 90, 91, 92, 93, 94, 95, 96, 97, 98, or 99%) identical to a reference standard consisting of a corresponding wild type IL-15 joined, in the same manner and orientation as the mutant IL-15, to a wild type Fc region. The mutation(s) in the antagonist can be within the Fc region as well as within the IL-15 polypeptide. For example, as shown in FIG. 3, the Fc region can include a mutation of the first glutamine residue and the first cysteine residue (in FIG. 3, the sequence EPKSCD (SEQ ID NO:27) is mutated to DPKSAD (SEQ ID NO:28). In the antagonists described herein, the Fc region can be a human Fcγ1 domain having either or both of these mutations. Antagonists that include, or that consist of, a mutant IL-15 polypeptide and an Fc region can: (a) include a mutation at position 101 and/or position 108 of SEQ ID NO:6 and a mutation within the Fc region (e.g., a mutation at position 115 and/or 119 of SEQ ID NO:6), (b) exhibit at least 90% identity to a corresponding polypeptide that includes, or that consists of, the corresponding wild type IL-15 and Fc regions, and (c) inhibit one or more of the activities mediated by wild type IL-15 (e.g., signal transduction through the IL-15R).

In one embodiment, the Fc region is a mutated human IgG1 Fc region comprising, or consisting of, the following sequence:

(SEQ ID NO:_)
DPKSADKTHTCPPCPAPELLGGPSVFLFPPKPKDTLMISRTPEVTCVVVDVSHEDP
EVKFNWYVDGVEVHNAKTKPREEQYNSTYRVVSVLTVLHQDWLNGKEYKCKV
SNKALPAPIEKTISKAKGQPREPQVYTLPPSRDELTKNQVSLTCLVKGFYPSDIAV
EWESNGQPENNYKTTPPVLDSDGSFFLYSKLTVDKSRWQQGNVFSCSVMHEAL
HNHYTQKSLSLSPGK.

The percentage of identity between a subject sequence and a reference standard can be determined by submitting the two sequences to a computer analysis with any parameters affecting the outcome of the alignment set to the default position. In some instances (e.g., where any mutations are point mutations), a subject sequence and the reference standard can exhibit the required percent identity without the introduction of gaps into one or both sequences. In many instances, the extent of identity will be evident without computer assistance.

As illustrated by the statements above, the mutant IL-15 can differ from a corresponding wild type IL-15 (e.g., a mutant human IL-15 can differ from a wild type human IL-15) by one or more deletions, insertions, or amino acid substitutions, whether the substitutions represent conservative or non-conservative amino acid substitutions, in any part or region of the polypeptide, including the carboxy-terminal domain, which is believed to bind the IL-2Rα subunit (e.g., the residues LLELQVISL (SEQ ID NO:__) or the residues ENLII (SEQ ID NO:__); see Bernard et al., J. Biol. Chem. 279:24313-24322, 2004). One or more mutations can also be introduced within the IL-2Rγ binding domain or the IL-2Rβ binding domain. As noted above, the mutant polypeptides described herein that include all or part of an Fc region are polypeptides of the invention, even when not fused or otherwise joined to another polypeptide or when fused or otherwise joined to another polypeptide such as IL-15 or another therapeutic polypeptide, whether mutant or wild type.

A mutant IL-15 polypeptide, whether alone or as a part of a chimeric polypeptide or other protein complex, can be encoded by a nucleic acid molecule, including a molecule of genomic DNA, cDNA, or synthetic DNA. Any desired mutation can be introduced into a corresponding wild type IL-15 gene sequence by molecular biology techniques well known in the art. Just as the mutant IL-15-containing polypeptides can be described as having a certain “percent identity” with a corresponding wild type protein (a reference standard), the nucleic acid molecules encoding them can be described as having a certain “percent identity” with a corresponding wild type nucleic acid sequence. The nucleic acid molecules can also be characterized in terms of the polypeptides they encode. For example, a nucleic acid molecule within the scope of the present invention can encode a polypeptide that exhibits a certain minimal amount of identity to a reference polypeptide. For example, a nucleic acid molecule can encode a polypeptide that is at least or about 90% (e.g., 89, 90, 91, 92, 93, 94, 95, 96, 97, 98, or 99%) identical to a reference standard consisting of a corresponding polypeptide (e.g., a wild type IL-15 polypeptide).

The polypeptides of the invention can be, but are not required to be, substantially free of heterologous biological agents. Where a polypeptide is substantially pure, it can be at least about 50% (e.g., at least about 55, 60, 65, 70, 75, 80, 85, 90, 95, 97, or 99% pure). As noted, purity can be assessed with respect to heterologous biological agents, which include non-IL-15 polypeptides, other proteins, and cellular material such as lipids and nucleic acids. A substantially pure mutant IL-15 polypeptide may be one that is isolated from a cell that expresses a recombinant nucleic acid encoding the mutant IL-15 polypeptide or one that is chemically synthesized. Purity can be measured by any appropriate method, including column chromatography, polyacrylamide gel electrophoresis, or HPLC analysis.

While the invention is not limited to polypeptides that function by any particular mechanism, the IL-15 mutants (or variants thereof (e.g., chimeric proteins containing an IL-15 mutant)) may bind the IL-15 receptor complex with an affinity that is comparable to that of the wild type IL-15 (e.g. a mature human IL-15). Further, the mutant IL-15 may not activate the receptor as wild type IL-15 would (or may not activate it as fully). Therefore, the mutant IL-15 polypeptide can be used to block the receptor, and we may therefore refer to the mutant IL-15 polypeptides as IL-15 antagonists. The degree of receptor blockade can vary. What matters is that mutant IL-15 polypeptides employed as antagonists compete with wild type IL-15 to an extent that confers a benefit upon a patient to whom the mutant is administered.

The mutant IL-15/Fc polypeptides described herein can be dimerized, and such dimers are within the scope of the present invention. The dimer can consist of two identical polypeptides, which we may refer to as homodimers, or two non-identical polypeptides, which we may refer to as heterodimers. For example, a homodimer of the invention can include two copies of the polypeptide represented by SEQ ID NO:7, and a heterodimer of the invention can include only one copy of the polypeptide represented by SEQ ID NO:7. Regardless of the precise polypeptides used, the C-termini and N-termini can be aligned or roughly aligned. For example, where each of the polypeptides includes an Fc region at the N-terminus, the dimer can include molecular bonds between the two Fc regions (e.g., disulfide bonds between one or more of the cysteine residues within one Fc region and the other).

In some embodiments, a mutant IL-15 polypeptide described herein is conjugated to a water-soluble polymer, e.g., to increase stability or circulating half life or reduce immunogenicity. Clinically acceptable, water-soluble polymers include, but are not limited to, polyethylene glycol (PEG), polyethylene glycol propionaldehyde, carboxymethylcellulose, dextran, polyvinyl alcohol (PVA), polyvinylpyrrolidone (PVP), polypropylene glycol homopolymers (PPG), polyoxyethylated polyols (POG) (e.g., glycerol) and other polyoxyethylated polyols, polyoxyethylated sorbitol, or polyoxyethylated glucose, and other carbohydrate polymers. Methods for conjugating polypeptides to water-soluble polymers such as PEG are described, e.g., in U.S. Pat. Pub. No. 20050106148 and references cited therein.

Nucleic acids, vectors, and modified cells: The mutant IL-15 polypeptide, either alone or as a part of the chimeric polypeptides described above, can be encoded by a nucleic acid molecule, including a molecule of genomic DNA, cDNA, or synthetic DNA, and such nucleic acids are within the scope of the present invention. Similarly, the mutant Fc regions described herein, either alone or as a part of the chimeric polypeptides described above (those that include a mutant IL-15 and those that do not) are also within the scope of the present invention. As the nucleic acid molecules encode mutant and/or chimeric polypeptides, they are not expected to be found in nature. Nevertheless, the nucleic acids may be formulated in a manner that can be described as substantially pure. For example, a substantially pure nucleic acid molecule of the invention can be separated from other nucleic acid molecules and/or separated from other biological molecules.

The sequences of the nucleic acid molecules can vary due to the degenerate nature of the genetic code, and degenerate variants are within the scope of the present invention.

The nucleic acid molecules encoding a mutant IL-15 may be contained within a vector that is capable of directing expression of the IL-15 polypeptide in, for example, a cell that contains the vector (e.g., a cell that has been transduced with the vector). The vectors can be viral vectors (e.g., a retroviral, adenoviral, or adenoviral-associated vector), as well as plasmids or cosmids. Suitable vectors include T7-based vectors for use in bacteria (see, e.g., Rosenberg et al., Gene 56:125,1987), the pMSXND expression vector for use in mammalian cells (Lee and Nathans, J. Biol. Chem. 263:3521, 1988), and baculovirus-derived vectors (for example, the expression vector pBacPAK9 from Clontech, Palo Alto, Calif., USA) for use in insect cells. While additional promoters are described elsewhere, we note that a T7 promoter can be used when the host cells are bacterial, and a polyhedron promoter can be used in insect cells.

Yeast vectors typically contain an origin of replication sequence, an autonomously replicating sequence (ARS), a promoter region, sequences for polyadenylation, sequences for transcription termination, and a selectable marker. Suitable promoter sequences for yeast vectors include promoters for metallothionein, 3-phosphoglycerate kinase (Hitzeman et al., J. Biol. Chem. 255:2073, 1980) or other glycolytic enzymes (Hess et al., J. Adv. Enzyme Reg. 7:149, 1968; and Holland et al., Biochem. 17:4900, 1978), such as enolase, glyceraldehyde-3-phosphate dehydrogenase, hexokinase, pyruvate decarboxylase, phosphofructokinase, glucose-6phosphate isomerase, 3-phosphoglycerate mutase, pyruvate kinase, triosephosphate isomerase, phosphoglucose isomerase, and glucokinase. Other suitable vectors and promoters for use in yeast expression are further described in Hitzeman, EP-A-73,657.

Mammalian expression vectors typically include nontranscribed regulatory elements such as an origin of replication, a promoter sequence, an enhancer linked to the structural gene, other 5′ or 3′ flanking nontranscribed sequences (e.g., ribosome binding sites, a polyadenylation site, splice donor and acceptor sites, and transcriptional termination sequences). Regulatory sequences derived from Polyoma, Adenovirus 2, Simian Virus 40 (SV40), and human cytomegalovirus are frequently used for recombinant expression in mammalian cells. For example, SV40 origin, early and late promoter, enhancer, splice, and polyadenylation sites may be used to provide the other genetic elements required for expression of an IL-15 mutant DNA sequence in a mammalian host cell. Cytomegalovirus or metallothionein promoters are also frequently used in mammalian cells.

Prokaryotic (e.g., bacterial cells such as E. coli cells) or eukaryotic cells (e.g., yeast cells, or mammalian cells such as CHO cells) that contain and express nucleic acids encoding any of the mutant IL-15 polypeptides are also features of the invention. For yeast expression, cells of the Saccharomyces genus (e.g., S. cerevisiae) may be used. Alternatively, cells of other genera of yeast, such as Pichia or Kluyveromyces, may be used.

Examples of suitable mammalian host cell lines for production of mutant IL-15 polypeptides include: CHO cells; COS cell lines derived from monkey kidney, (e.g., COS-7 cells, ATCC number CRL 1651); L cells; C127 cells; 3T3 cells (ATCC number CCL 163); HeLa cells (ATCC number CCL 2); and BHK (ATCC number CRL 10) cell lines.

The method of transduction, the choice of expression vector, and the host cell may vary. The components of the expression system are compatible with one another, a determination that is well within the abilities of skilled artisans. Furthermore, for guidance in selecting an expression system, skilled artisans may consult Ausubel et al. (Current Protocols in Molecular Biology, John Wiley and Sons, New York, N.Y., 1993) and Pouwels et al. (Cloning Vectors. A Laboratory Manual, 1987).

Methods of treatment: Through the administration of a lytic form of the mutant IL-15 polypeptide, it is possible to selectively kill autoreactive or “transplant destructive” immune cells without massive destruction of normal T cells. Accordingly, the invention features methods of suppressing the immune response in a patient by administering a dose of mutant IL-15 sufficient to competitively bind the IL-15 receptor complex and thereby modulate (e.g., inhibit) the immune response. Alternatively, or in addition, one can administer a nucleic acid that encodes the mutant IL-15 polypeptide or a cell that expresses it. The polypeptide administered, the nucleic acid encoding it, or a cell expressing it, may be any of the mutant IL-15 polypeptides, nucleic acids, and cells described above, including those in which the mutant IL-15 is a part of a chimeric polypeptide (e.g., the chimeric polypeptide represented by SEQ ID NO:7).

These methods can be used to treat a patient who is suffering from an autoimmune disease, including but not limited to the following: (1) a rheumatic disease such as rheumatoid arthritis, systemic lupus erythematosus, Sjögren's syndrome, scleroderma, mixed connective tissue disease, dermatomyositis, polymyositis, Reiter's syndrome or Behcet's disease (2) type II diabetes (3) an autoimmune disease of the thyroid, such as Hashimoto's thyroiditis or Graves' Disease (4) an autoimmune disease of the central nervous system, such as multiple sclerosis, myasthenia gravis, or encephalomyelitis (5) a variety of phemphigus, such as phemphigus vulgaris, phemphigus vegetans, phemphigus foliaceus, Senear-Usher syndrome, or Brazilian phemphigus, and (6) psoriasis. The administration of the mutant IL-15 polypeptide of the invention may also be useful in the treatment of acquired immune deficiency syndrome (AIDS). Similarly, the method may be used to treat a patient who has received a transplant of biological materials, such as an organ, tissue, or cell transplant. In addition, there is reason to believe that patients who have received a vascular injury would benefit from this method.

The invention also features a method of inhibiting the growth of malignant cells that express the IL-15 receptor in vivo. This method is carried out by administering to a patient an amount of mutant IL-15 linked to a polypeptide that is sufficient to activate the complement system, lyse cells by the ADCC mechanism, or otherwise kill cells expressing the wild-type IL-15 receptor complex. In some specific embodiments, the patient may be suffering from a leukemia or lymphoma. As with the other types of patients described above, those having a malignant growth can be treated with the mutant IL-15 polypeptide, a nucleic acid molecule that encodes it, or a cell that expresses it (e.g., the therapy can be a gene-based or cell-based therapy).

The polypeptide of the invention may also be used to diagnose a patient as having a disease amenable to treatment with an IL-15 antagonist. According to this method, a sample of tissue is obtained from the patient and exposed to an antigenically-tagged mutant IL-15 polypeptide. The sample may be any biological sample. Preferably, the sample is a blood, serum, or plasma sample, but it may also be a urine sample, a tissue sample (e.g., biopsy tissue), or an effusion obtained from a joint (e.g. synovial fluid), from the abdominal cavity (e.g., ascites fluid), from the chest (e.g., pleural fluid), from the central nervous system (e.g., cerebral spinal fluid), or from the eye (e.g., aqueous humor). The sample may also consist of cultured cells that were originally obtained from a patient (e.g., peripheral blood mononuclear cells). It is expected that the sample will be obtained from a mammal, and preferably, that the mammal will be a human patient. If the sample contains cells that are bound by the polypeptide described, it is highly likely that they would be bound by mutant IL-15 polypeptide in vivo and thereby inhibited from proliferating in vivo. The presenting symptoms of candidate patients for such testing and the relevant tissues to be sampled given a particular set of symptoms are known to those skilled in the field of immunology.

In therapeutic applications, the polypeptide may be administered with a physiologically-acceptable carrier, such as physiological saline by any standard route including intraperitoneally, intramuscularly, subcutaneously, or intravenously. As other polypeptide-based therapies are executed via intravenous administration, it is expected that the intravenous route will be preferred. It is well known in the medical arts that dosages for any one patient depend on many factors, including the general health, sex, weight, body surface area, and age of the patient, as well as the particular compound to be administered, the time and route of administration, and other drugs being administered concurrently. Dosages for the polypeptide of the invention will vary, but a preferred dosage for intravenous administration is approximately 0.01 mg to 100 mg/kg (e.g., 0.01-1 mg/kg). Determination of correct dosage for a given application is well within the abilities of one of ordinary skill in the art of pharmacology.

### EXAMPLES

In the studies described below, we found that administration of a lytic and antagonistic IL-15 mutant/Fcγ2a fusion protein prevented rejection and induced antigen-specific tolerance of minor histocompatibility complex-mismatched grafts in a B10.Br to CBA/Ca strain combination and prolonged the survival of transplanted hearts in fully MHC-mismatched recipients in a Balb/c to C57BL/6 mouse strain combination. Prolonged graft survival was accompanied by reduced mononuclear cell infiltration and inflammatory cytokine expression in the treated graft recipients.

Generation of mIL-15/Fc chimeric proteins: cDNA for Fcγ2a can be generated from mRNA extracted from an IgG2a secreting hybridoma using standard techniques with reverse transcriptase (MMLV-RT; Gibco-BRL, Grand Island, N.Y.) and a synthetic oligo-dT (12-18) oligonucleotide (Gibco BRL). The mutant IL-15 cDNA can be amplified from a plasmid template by PCR using IL-15-specific synthetic oligonucleotides. For example, the 5′ oligonucleotide is designed to insert a unique NotI restriction site 40 nucleotides 5′ to the translational start codon, while the 3′ oligonucleotide eliminates the termination codon and modifies the C-terminal Ser residue codon usage from AGC to TCG to accommodate the creation of a unique BamHI site at the mutant IL-15/Fc junction. Synthetic oligonucleotides used for the amplification of the Fcγ2a domain cDNA change the first codon of the hinge from Glu to Asp in order to create a unique BamHI site spanning the first codon of the hinge and introduce a unique XbaI site 3′ to the termination codon. The Fc fragment can be modified so that it is non-lytic (i.e., not able to activate the complement system). To make the non-lytic mutant IL-15 construct (we may refer to the non-lytic mutant as “mIL-15/Fc−−”), oligonucleotide site directed mutagenesis is used to replace the C1q binding motif Glu³¹⁸, Lys³²⁰, Lys³²² with Ala residues. Similarly, Leu²³⁵ is replaced with Glu to inactivate the FcγRI binding site. Ligation of cytokine and Fc components in the correct translational reading frame at the unique BamHI site yields a 1236 bp open reading frame encoding a single 411 amino acid polypeptide (including the 18 amino acid IL-15 signal peptide) with a total of 13 cysteine residues. The mature secreted homodimeric IL-15/Fc−− is predicted to have a total of up to eight intramolecular and three inter-heavy chain disulfide linkages and a molecular weight of approximately 85 kD, exclusive of glycosylation.

Expression and Purification of mIL-15 Fc Fusion Proteins: Proper genetic construction of both mIL-15/Fc++, which carries the wild type Fcγ2a sequence, and mIL-15/Fc−− can be confirmed by DNA sequence analysis following cloning of the fusion genes as NotI-XbaI cassettes into the eukaryotic expression plasmid pRc/CMV (Invitrogen, San Diego, Calif.). This plasmid carries a CMV promoter/enhancer, a bovine growth hormone polyadenylation signal and a neomycin resistance gene for selection with G418 (of course, many other plasmids are suitable as expression vectors). Plasmids carrying the mIL-15/Fc++ or mIL-15/Fc−− fusion genes can be transfected into Chinese hamster ovary cells (CHO-K1, available from the American Type Culture Collection) by electroporation (1.5 kV/3 μF/0.4 cm/PBS) and selected in serum-free Ultra-CHO™ media (Bio Whittaker Inc., Walkerville, Md.) containing 1.5 mg/ml of G418 (Geneticin, Gibco BRL). After subcloning, clones that produce high levels of the fusion protein can be selected by screening supernatants for IL-15 by ELISA (PharMingen, San Diego, Calif.). mIL-15/Fc fusion proteins are purified from culture supernatants by protein A Sepharose™ affinity chromatography followed by dialysis against PBS and 0.22 μm filter sterilization. Purified proteins can be stored at −20° C. before use.

Western blot analysis following SDS-PAGE under reducing (with DTT) and non-reducing (without DTT) conditions can be performed using monoclonal or polyclonal anti-mIL-15 or anti Fcγ primary antibodies to evaluate the size and isotype specificity of the fusion proteins.

In studies of another cytokine, IL-2, we found that molecular weight (MW) measured by proteomic analysis could vary, depending upon the host cell type. The MW of IL-2/Fc produced by CHO cells was 94,838.7, while the same molecule produced in NS.1 cells was only 91,647.5. Differences in glycosylation may account for the difference in MW. Further, the difference in glycosylation appears to influence function, as IL-2/Fc molecules produced in CHO cells suppressed the development of diabetes in non-obese diabetic mice more effectively than the same molecule produced in NS.1 cells.

Standardization of the Biological Activity of Recombinant Mutant IL-15 and mIL-15/Fc−− proteins: Using the RT-PCR strategy and 5′ NotI sense oligonucleotide primer described above, mutant IL-15 cDNA with an XbaI restriction site added 3′ to its native termination codon, can be cloned into pRc/CMV. This construct can then be transiently expressed in COS cells (available from the American Type Culture Collection). The cells may be transfected by the DEAE dextran method and grown in serum-free UltraCulture™ media (Bio Whittaker Inc.). Day 5 culture supernatant is sterile filtered and stored at −20° C. for use as a source of recombinant mutant IL-15 protein (rmIL-15). Mutant IL-15/Fc−− and mIL-15 mutant protein concentrations can be determined by ELISA as well as by bioassay, as described, for example, by Thompson-Snipes et al. (J. Exp. Med. 173:507, 1991). Dual probe ELISA assays, which are useful here, are quantitative “sandwich” enzyme immunoassays. In one study, we coated microtiter plates with rat IgG antibodies specific for mouse/human IL-15. Test samples of IL-15/Fc were added to the wells, and unbound components in the sample were washed away. Enzyme-linked rabbit antibodies specific for mouse IgG2a Fc/human IgG1 Fc were then added to the wells, creating a sandwich, with IL-15/Fc bound by the coated anti-IL-15 antibody and the anti-mouse IgG2a Fc/human IgG1 Fc antibody. Such dual probe ELISAs ensure the assay is specific for mouse IL-15/Fc fusion protein (rather than IL-15 or mGgG2a/hIgG1. Excess enzyme-conjugated IgG can be removed by washing before the enzyme substrate is added to the wells. A colored reaction product developes in proportion to the amount of IL-15/Fc present in the sandwich.

The functional activity of mutant IL-15/Fc−− can be assessed by a standard T cell proliferation assays, such as those described in U.S. Pat. No. 6,451,308.

Determination of mIL-15/Fc−− or mIL-15/Fc++ Circulating Half-life: Serum concentrations of mIL-15/Fc−− or mIL-15/Fc++ fusion proteins can be determined over time following a single intravenous injection of the fusion protein (non-fusion proteins can be similarly assessed). Serial 100 μl blood samples can be obtained by standard measures at intervals of, for example, about 0.1, 6.0, 24.0, 48.0, 72.0, and 96.0 hours after administration of mutant IL-15/Fc−− protein. Measurements employ an ELISA with a mIL-15 mAb as the capture antibody and horseradish peroxidase conjugated to an Fcγ2a mAb as the detection antibody, thus assuring this assay is specific for only the mutant IL-15/Fc−−.

Procedures for Screening Mutant IL-15 Polypeptides: One or more of the following transplantation paradigms and models of autoimmune disease can be employed to determine whether any given polypeptide (e.g., any given mutant IL-15 polypeptide) is capable of functioning as an antagonist of IL-15.

Mutant IL-15 polypeptides can be administered, directly or by genetic therapy, in the context of well-established transplantation paradigms. For example, a putative immunosuppressing polypeptide, or a nucleic acid molecule encoding it, could be systemically or locally administered by standard means to any conventional laboratory animal, such as a rat, mouse, rabbit, guinea pig, or dog, before an allogeneic or xenogeneic skin graft, organ transplant, or cell implantation is performed on the animal. Strains of mice such as C57B1-10, B10.BR, and B10.AKM (Jackson Laboratory, Bar Harbor, Me.), which have the same genetic background but are mismatched for the H-2 locus, are well suited for assessing various organ grafts.

A method for performing cardiac grafts by anastomosis of the donor heart to the great vessels in the abdomen of the host was first published by Ono et al. (J. Thorac. Cardiovasc. Surg. 57:225, 1969; see also Corry et al., Transplantation 16:343, 1973). According to this surgical procedure, the aorta of a donor heart is anastomosed to the abdominal aorta of the host, and the pulmonary artery of the donor heart is anastomosed to the adjacent vena cava using standard microvascular techniques (this procedure was used in the studies described below). Once the heart is grafted in place and warmed to 37° C. with Ringer's lactate solution, normal sinus rhythm will resume. Function of the transplanted heart can be assessed frequently by palpation of ventricular contractions through the abdominal wall. Rejection is defined as the cessation of myocardial contractions, which can be confirmed by under anesthesia. Mutant IL-15 polypeptides would be considered effective in reducing organ rejection (or prolonging graft survival) if hosts that received injections, for example, of the polypeptide tolerated the grafted heart longer than did untreated hosts.

The effectiveness of mutant IL-15 polypeptides can also be assessed following a skin graft. To perform skin grafts on a rodent, a donor animal is anesthetized and the full thickness skin is removed from a part of the tail. The recipient animal is also anesthetized, and a graft bed is prepared by removing a patch of skin from the shaved flank. Generally, the patch is approximately 0.5×0.5 cm. The skin from the donor is shaped to fit the graft bed, positioned, covered with gauze, and bandaged. The grafts can be inspected daily beginning on the sixth post-operative day, and are considered rejected when more than half of the transplanted epithelium appears to be non-viable.

Models of autoimmune disease provide another means to assess polypeptides in vivo. These models are well known to skilled artisans and can be used to determine whether a given mutant IL-15 polypeptide is an immunosuppressant that would be therapeutically useful in treating a specific autoimmune disease when delivered to a patient (e.g., via genetic therapy).

The following materials and methods were used in the studies described below and can be used in connection with the compositions and methods described herein (for example, the animals and transplantation paradigms can be used in pre-clinical analysis of IL-15R antagonists, including mIL-15/Fc fusion proteins).

Animals: BALB/c (H-2d) and C57BL/6 (H-2b) mice, 8-10 weeks old, were purchased from Charles River Laboratories (Wilmington, Mass.). B 10.A (H-2d), CBA/Ca (H-2k), B10.BR (H-2k) and AKR/J (H-2k) mice were obtained from The Jackson Laboratory (Bar Harbor, Me.).

Reagents and Treatment Protocols: A construct for expressing a lytic and antagonistic IL-15 mutant/Fcγ2a fusion protein was designed and constructed as described by Kim et al. (J. Immunol. 160:5742, 1998). Briefly, glutamine residues 101 and 108 within the fourth alpha helix of IL-15 were mutated to asparatic acid via site-directed and PCR-assisted mutagenesis. This mutant IL-15 was then genetically linked to the hinge and constant regions of murine IgG2a and further cloned into an expression vector. NS-1 cells (obtained from the American Type Culture Collection (ATCC), Manassas, Va.) or CHO-K1 cells (DMSZ, Braunschweig, Germany), were stably transfected with a plasmid carrying the construct encoding the fusion protein (Kim et al., J. Immunol. 160:5742, 1998). The transfected cells were cloned and cultured in serum-free Ultraculture™ media (Bio Whittaker Inc, Walkersville, Md.) containing 100 μg/ml Zeocin (Invitrogen, San Diego, Calif.). Expressed protein (IL-15/Fc) in the culture supernatant was purified by Protein A affinity chromatography and, in some instances, ion-exchange chromatography. A non-lytic IL-15 mutant/Fcγ2a fusion construct was generated essentially as described by Zheng et al. (see Zheng et al., J. Immunol. 163:4041, 1999, and Zheng et al., 1997, J. Immunol. 158:4507, 1997).

Briefly, oligonucleotide site-directed mutagenesis was used to replace the IgG2a C1q binding motif Glu318, Lys320, Lys322 with Ala residues. Similarly, the IgG2a residue Leu235 was replaced with Glu to inactivate the FcγRI binding site (see Zheng et al., J. Immunol. 163:4041, 1999, and Zheng et al., J. Immunol. 158:4507, 1997).

A monoclonal antibody against CD154 (MR-1, IgG2a) was obtained from Chimerigen Laboratories (Allston, Mass.). Heart and islet allograft recipients were treated daily or every second day with 1.5 μg, 5 μg or 15 μg of the fusion protein by intraperitoneal injection or with 15 μg of control (IgG2a, also administered intraperitoneally) for a total of 14 days. The first treatment was given on the day of transplantation, after the surgical procedure. Treatment with anti-CD154 (anti-CD40L) was with a single dose of 200 μg administered intraperitoneally on the day of transplantation, also after surgery had been completed.

Heart Transplantation: Abdominal heterotopic heart transplants were performed essentially as described by Corry et al. (Transplantation 16:343, 1973). The isolated donor heart was grafted by joining the donor aorta to the recipient aorta and the donor pulmonary artery to the recipient vena cava. After an initial recovery period, animals bearing such transplants were housed under standard conditions, and we recorded the palpable heartbeat of the graft every 1 to 2 days. Animals were scored as having rejected the graft upon complete loss of palpable heartbeat. In some instances, animals with long term surviving grafts received a secondary cervical heart transplant. The basic procedures were identical to the ones used for abdominal aortic grafts, except that the second heart was grafted onto the carotid artery by side to end anastomosis with the aorta and side to end anastomosis of the pulmonary artery to the jugular vein. In all instances, 11-0 suture material was used for these procedures.

Islet transplantation: Islet transplantation was performed according to procedures described by Ferrari-Lacraz et al. (J. Immunol. 167;3478, 2001). Donor pancreata from 8-10 wk male Balb/c (H-2d) mice were perfused in situ with 4 ml Type IV collagenase (Worthington Biochemical Corp. Freehold, N.J.) through the common bile duct.

The pancreata were harvested after perfusion and incubated at 37° C. for 35 minutes. Islets were released from the pancreata by gentle vortexing and further purified on discontinuous percoll gradients, washed twice and 300 to 400 islets were transplanted under the left renal capsule of 8-10 wk old, completely MHC mismatched, C57BL/6 recipients rendered diabetic by a single intraperitoneal injection of streptozotocin (260 mg/kg in 0.9% NaCl; Sigma Chemical Co., St. Louis, Mo.). Allograft function was monitored by serial blood glucose measurements (Accu-Chek™ III blood glucose monitor; Boehringer Mannheim, Indianapolis, Ind.). Primary graft function was defined as a blood glucose level below 200 mg/dl on day 3 post-transplantation, and graft rejection was defined as a rise in blood glucose exceeding 300 mg/dl following a period of satisfactory primary graft function. To determine whether tolerance was evident in treated recipients, a nephrectomy was performed on islet allograft recipient mice with euglycemia for 120 days after primary transplantation. Removal of the left kidney bearing the islet allograft 120 days post-transplantation resulted in prompt hyperglycemia exceeding 300 mg/dl within 2-3 days. The second islet allografts from Balb/c or B10.A donors were transplanted under the right kidney capsule of hyperglycemic mice 4-6 days post nephrectomy. We monitored secondary graft function by measuring the blood glucose levels of the recipient mice as described above.

Histopathology and Immunochistochemistry: Transplanted hearts were harvested at Day 5 after transplantation and divided into three parts by cutting through the heart twice, perpendicular to the intraventricular septum. The first ⅓ of the tissue was fixed in zinc formalin for hematoxylin/eosin and immunohistochemistry (CD3 and F4/80 detection), and paraffin sections were prepared from these samples; the second ⅓ of the tissue was imbedded in OCT and snap-frozen in liquid nitrogen to −80° C. for immunohistochemistry (CD4 and CD8 detection); and the last ⅓ was analyzed by RT-PCR (see below). After dehydration and paraffin embedding, 5- to 6-μm-thick sections of the heart were stained with H&E. Multiple sections of each heart were prepared and examined for the extent of rejection, myocardial damage, mononuclear cell infiltration, vasculitis and intimal proliferation. The avidin-biotin immunoperoxidase method was used for immunohistochemistry. Images were obtained using an Axioscope™ 2 microscope (Zeiss) equipped with a digital camera (SV Micro 80155) and interfaced with image analysis software (KS 300). Quantitative image analysis was performed on ten random sections from each section of the heart stained for different cell markers (CD4 and CD8). Quantitative image analysis was performed on three hearts from the control group and three hearts from the treatment group. The number of positively stained cells and total area occupied by these cells were compared for CD4 and CD8 cell markers in hearts of treated and control animals.

For islet transplants, the left kidneys bearing islet allografts were removed from long-term graft accepting mice and processed further. In addition, transplant-bearing kidneys from C57B1/6 mice that had received Balb/c islet allografts were removed on Day 7 post-transplantation. The kidneys were fixed in zinc formalin for hematoxylin/eosin and aldehyde-fuchsin staining and immunohistochemistry (insulin detection); paraffin sections were prepared from the samples processed in this way, and 5- to 6-μm-thick sections of areas of islet implantation were stained. Multiple sections of each kidney were prepared and examined for islet content and insulin production. The avidin-biotin immunoperoxidase method was used for immunohistochemistry, and images obtained as described for heart transplants.

RNA isolation and reverse transcriptase assisted polymerase chain reaction (RT-PCR): Total cellular RNA was extracted using RNASTAT™ 60 (Tel Test, Friendswood, Tex.) according to the manufacturer's instructions. We checked the quality of the RNA by performing a PCR analysis to detect traces of chromosomal DNA, and we determined the concentration of the RNA using a Beckman Coulter Spectrophotometer DU 640. Two micrograms of RNA were reverse-transcribed and quality controlled for the expression of the housekeeping gene cyclophilin (Smith et al., J. Immunol. 165:3444, 2000). Subsequently, the relative abundance of the inflammatory cytokines (IL-1β, IL-6 and TNFα), IFNγ, and the CTL markers FasL, granzyme B and perforin were determined by TaqMan™ real-time PCR analysis with the ABI 7000 Sequence detection instrument and normalized against the housekeeping gene cyclophilin. Primers and probes for IL-1β, IL-6 and TNFα were purchased from Applied Biosystems, primers for cyclophilin (CYC), IFNγ (IFN), FasL (FSL), granzyme B (GRB) and perforin (PRF) were:

| | |
|---|---|
| CYCF: | |
| GCCTGGATGCTAACAGAAGGA; | (SEQ ID NO:_) |
| CYCR: | |
| GTTCATCCCGTCGCTATGGT; | (SEQ ID NO:_) |
| CYCprobe: | |
| ATGACAAGGATGCCGGGCAAGTGT; | (SEQ ID NO:_) |
| FSLF: | |
| AATCTGTGGCTACCGGTGGTA; | (SEQ ID NO:_) |
| FSLR: | |
| GGTGGAAGAGCTGATACATTCCTA; | (SEQ ID NO:_) |
| FSLprobe: | |
| ATGGTTCTGGTGGCTCTGGTTGGAA; | (SEQ ID NO:_) |
| GRBF: | |
| GCAAAGACTGGCTTCATATCCAT; | (SEQ ID NO:_) |
| GRBR: | |
| GCAGAAGAGGTGTTCCATTGG; | (SEQ ID NO:_) |
| GRBprobe: | |
| ACAAGGACCAGCTCTGTCCTTGGCAG; | (SEQ ID NO:_) |
| PRFF: | |
| TGCTCTTCGGGAACCAAGCT; | (SEQ ID NO:_) |
| PRFR: | |
| CAGGGTTGCTGGGCAGTGA; | (SEQ ID NO:_) |
| PRFprobe: | |
| CACCAGAGCAGTTCTCAACCTGGACAGC; | (SEQ ID NO:_) |
| IFNF: | |
| ACAATGAACGCTACACACTGCAT; | (SEQ ID NO:_) |
| IFNR: | |
| TGGCAGTAACAGCCAGAAACA; | (SEQ ID NO:_) |
| IFNprobe: | |
| TTGGCTTTGCAGCTCTTCCTCATGG. | (SEQ ID NO:_) |

Statistical analysis. Animal survival data were analyzed using a survival curve Logrank test as provided by Prism™ software (version 3.0). Histological data generated by Image Analysis were evaluated for statistical significance using Student's two-tailed t test at the 0.05 significance level. The Microsoft Excel data analysis tool was used to obtain mean and standard deviation as well as Student's t test results. We generated real-time PCR data by analyzing each cDNA sample in triplicate by TaqMan™ realtime PCR. Automatic baseline determination using the ABI 7000 Sequence detection instrument was followed by manual quality control. Primary data were processed in an Excel spreadsheet format and exported into the Prism software (version 3.0) for the graphical display. Data generated were evaluated for statistical significance using a Student's two tailed t test.

As noted above, we have found that administration of a lytic and antagonistic IL-15 mutant/Fcγ2a fusion protein can prevent rejection and induce antigen-specific tolerance of minor histocompatibility complex-mismatched grafts in a B10.Br to CBA/Ca strain combination. The fusion protein can also prolong the survival of transplanted hearts in fully MHC-mismatched recipients, as we demonstrated with a Balb/c to C57BL/6 mouse strain combination. Prolonged graft survival was accompanied by reduced mononuclear cell infiltration and inflammatory cytokine expression in the treated graft recipients. In addition, we found that administering the fusion protein in combination with a sub-optimal dose of anti-CD 154 (CD40L) antibody confers permanent heart allograft engraftment in a fully MHC-mismatched mouse strain combination. Moreover, we demonstrated an induction of antigen-specific tolerance in a fully MHC-mismatched islet transplant model.

To further characterize the mode of action, we performed parallel experiments employing an IL-15/Fc variant with a point-mutated non-lytic IgG2a Fc. These experiments demonstrated that the Fc portion of the molecule contributes to the overall efficacy of the molecule in vivo.

Prolonging the Survival of Fully MHC-Mismatched Heart Allografts.

We tested the efficacy of our lytic and antagonistic IL-15 mutant/Fcγ2a fusion protein in preventing the rejection of fully MHC-mismatched heterotopic heart transplants in the Balb/c (H-2d) to C57BL/6 (H-2b) mouse strain combination. Control animals rejected the transplants with an MST=7d (Table I). While recipient C57BL/6 mice treated with 1.5 μg daily (for 14 days) experienced a marginal prolongation of engraftment, treatment with 5 μg daily (again, for 14 days) resulted in a pronounced prolongation of graft survival (MST=26d). In contrast, treatment with 15 μg did not lead to a further prolongation of graft survival, and animals in this treatment group rejected their transplants with kinetics similar to the animals in the 5 μg dose group (Table I). Interestingly, treatment of transplant recipients with 5 μg, every second day for 14 days (8 administrations total), led to a further prolongation of graft survival with an MST=35d (Table I). Treatment with 5 μg every three days (5 administrations total) showed an accelerated rejection of the transplanted hearts, as compared to a daily or bi-daily treatment regimen (Table I).

TABLE I
Survival of Balb/c heart allografts in C57/BL6 recipients

| | | | Heart Graft Survival |
|---|---|---|---|
| Donor | Recipient | Treatment | (days) |
| Balb/c | C57/BL6 ^{b(H-2)} | Control IgG2a 15 μg/day | 6, 7, 7, 8, 8 |
| (H-2d) | | for 14 days | |
| Balb/c | C57/BL6 ^{b(H-2)} | Treated 1.5 μg/day | 9, 10, 12, 12, 13 |
| (H-2d) | | for 14 days | p < 0.005^{a} |
| Balb/c | C57/BL6 ^{b(H-2)} | Treated 5 μg/day | 20, 22, 26, 30, 30 |
| (H-2d) | | for 14 days | p < 0.005^{a} |
| Balb/c | C57/BL6 ^{b(H-2)} | Treated 15 μg/day | 19, 22, 25, 28, 30 |
| (H-2d) | | for 14 days | p < 0.005^{a} |
| Balb/c | C57/BL6 ^{b(H-2)} | Treated 5 μg/2 days | 29, 32, 35, 35, 36 |
| (H-2d) | | for 14 days | p < 0.05^{b} |
| Balb/c | C57/BL6 ^{b(H-2)} | Treated 5 μg/3 days | 10, 12, 13, 14, 14 |
| (H-2d) | | for 14 days | p < 0.005^{b} |

^{a}Graft survival prolongation in IL-15-treated versus control-treated animals is statistically significant (logrank test; p < 0.005);
^{b}Graft survival versus animals treated every day

To assess the effect of treatment on graft rejection, we studied the graft cellularity in heart allografts harvested 5 days post-transplantation. The overall graft cellularity in treated mice was reduced compared to the control group. The inflammatory infiltrates in these hearts were focal, less numerous, and smaller than in the control-treated animals and ischemic myocardial cell damage with interstitial edema and hemorrhages was also strongly reduced in the treated animals. Vascular changes consisting of vasculitis and vascular endothelial cell proliferation and occlusion were also more evident in the control group than in the allografts of treated animals. A quantitative image analysis performed on these samples revealed a particularly striking reduction of leukocyte infiltration for CD8⁺ T cells, which was at 93.5% (n=3, p=0.008). Immunohistological detection of leukocyte subsets on day 5 showed a strongly reduced number of CD3⁺, CD4⁺, CD8⁺, and F4/80⁺ comparison, CD4⁺ T cells in the treated grafts were reduced by 58% (n=3, p<0.05).

To further study the effects of treatment on allogeneic transplant rejection, a real time PCR analysis on various inflammatory cytokines (IL-1β and TNFα), CTL effector molecules (FasL, Granzyme B and Perforin) and Th1/Th2 cytokines (IL-4 and IFNγ) was performed 5 days post transplantation. Whereas the expression of all of these markers was elevated in rejecting heart allografts of control-treated animals (C), treatment (T) led to a statistically significant reduction of expression of most of these genes in the transplanted hearts, with the notable exception of the Th2 cytokine IL-4. Similar results such as for IL-4 were also obtained for IL-5. Interestingly, a reduction in IL-10 expression was also observed in the treated grafts (p<0.001), likely reflecting the strong reduction in macrophages seen in the treated grafts.

The Contribution of the Fc Portion

Earlier studies have demonstrated that the deletion of antigen-specific T cells contributes to long-term engraftment and tolerance induction in various allograft settings (Li et al., Nat. Med. 5:1298, 1999; Wells et al., Nat. Med. 5:1303, 1999). To further characterize the mode of action and to directly investigate the potential contribution of the IgG2a Fc terminus to the overall efficacy, we generated a non-lytic point-mutated variant of the fusion protein described above that does not interact with complement or Fc receptors. Whereas a short course treatment with the lytic form leads to prolonged heart allograft survival (MST=25 days) in the Balb/c to C57/BL6 mouse strain combination, transplants in animals treated with the non-lytic variant are rejected with kinetics comparable to control-treated animals (MST=7 days).

Permanent Engraftment of MHC-Mismatched Allografts After Treatment with an IL-15/Fc and a Single Dose of Anti-CD154 Antibody.

While treatment with a lytic and antagonistic IL-15 mutant/Fcγ2a fusion protein prolongs heart allograft survival in MHC-mismatched recipients, the transplants are eventually rejected (Table I). As we have shown that treatment can prevent costimulation blockade resistant rejection in islet transplant models (Ferrari-Lacraz et al., J. Immunol. 167:3478, 2001), we were interested in determining whether blockade of the CD40/CD154 costimulation pathway would synergize with the fusion protein in preventing heart allograft rejection. Whereas treatment with a single dose of the antiCD154 monoclonal antibody MR-1 prolonged heart transplant survival in the Balb/c to C57/BL6 mouse strain combination, this treatment was insufficient to prevent rejection. In contrast, treatment with IL-15/Fc (5 μg/mouse every 2nd day) for 14 days, in combination with a single dose administration of the antiCD 154 antibody, was sufficient to prevent graft rejection in all animals tested (n=5) and led to permanent engraftment of the transplanted hearts.

Induction of Antigen-Specific Tolerance.

To further explore the therapeutic potential of IL-15/Fc, its efficacy in preventing the rejection of heterotopic heart transplants in a minor histocompatibility mismatch strain combination was tested by transplanting hearts from B10.BR to CBA/Ca mice. Treatment with 5 μg administered every second day for 14 days led to permanent engraftment of the transplanted hearts in this mouse strain combination. Control hearts were all rejected within 13 days after transplantation (MST=10 days). To test for antigen-specific tolerances, the CBA.Ca mice having received B10.BR allografts, and having been treated with IL-15/Fc received secondary heart allografts after prolonged survival of the primary grafts (>I 00d). These secondary heart transplants were from either B10.BR mice or from the third party strain AKR.J. Whereas the secondary grafts from the B10.BR donors were accepted without any further immunosuppression, the grafts from the AKR.J mice were efficiently rejected.

Similarly, the lytic fusion protein proved efficacious in preventing the rejection of islet allografts transplanted under the kidney capsule of streptozotocin-induced diabetic mice in the fully MHC-mismatched Balb/c to C57/BL6 strain combination. Treatment with 5 μg administered every second day for 14 days prolonged islet allograft survival and permanent engraftment in 50% of the treated animals. Seven days after transplantation, a strong reduction in islet cell mass and insulin-producing cells was apparent in untreated animals, as compared to the treated mice. 120 days after transplantation, the graft containing kidneys were removed from the treated animals with permanent engraftment and grafted islets in these animals were found to be preserved and functional, as determined by aldehyde-fuchsin and insulin staining. All animals examined became diabetic after removal of the grafts. Subsequently, these animals received a second islet graft under the capsule of the second kidney. Whereas mice receiving islets from Balb/c donors became normoglycemic, and remained so without any further treatment, a B10.A derived graft was rejected. We conclude that this treatment protocol can lead to antigen-specific tolerance and that monotherapy has the potential to induce tolerance also in a fully MHC-mismatched allograft setting.

The present studies extend prior observations by showing that IL-15IFc treatment also prolongs the graft survival of fully MHC-mismatched vascularized heart transplants. We find that treatment reduces the graft infiltration by CD4⁺ and CD8⁺ T cells as well as macrophages. The effect of the treatment is particularly striking for CD8⁺ T cells, in that CD8⁺ T cells are almost completely absent from the grafts of treated animals. In comparison, the effect on CD4⁺ T cells appears to be more moderate, a finding that is not surprising in view of earlier reports that IL-15 acts preferentially on CD8⁺ T cells, at least in IL-15 and IL-15Rα knockout systems (Lodolce et al., Immunity 9:669, 1998; Kennedy et al., J. Exp. Med. 191:771, 2000).

Consistent with the immunohistology results, we find that treatment reduces the expression of CTL markers in the grafts, as well as the expression of the inflammatory cytokines TNFα and IL-1β. Interestingly, whereas IL-15/Fc treatment leads to a reduction of Th1 cytokine expression (IFNγ and TNFα), no effect of treatment is seen on the expression of the Th2 cytokines IL-4 and IL-5. These data indicate that IL-15 may preferentially stimulate Th1 responses, further underlining the utility of IL-15 antagonistic approaches in targeting Th1-mediated diseases, such as many autoimmune disorders and graft rejection. The dose titration experiments performed in the Balb/c to C57/BL6 mouse strain combination revealed a dose response relationship and a direct correlation between the dose administered and the efficacy of the treatment. Interestingly, treatment every second day showed an increased efficacy as compared to a daily treatment and further delayed graft rejection. Accordingly, methods in which treatment is given on alternating days is within the scope of the present invention. Although not further examined, one possible explanation for this observation would be that IL-15/IL-15R signaling within the tissue might be protective under conditions of ischemia and/or reperfusion, such as in the initial periods post surgery.

The reduced efficacy we observed with administration only once every three days, on the other hand, is consistent with the observed half-life of the molecule in mice, which is about 30 hours.

We have previously demonstrated that the deletion of activated T cells can contribute to peripheral tolerance induction, suggestive of the notion that depletion of the pool of antigen-responsive T cells may shift the balance of an immune reaction from an immunogenic to a tolerogenic response (Li et al., Immunity 14:407, 2001). In view of these earlier findings we were interested in determining whether the IgG2a Fc portion of the fusion protein would contribute to the overall efficacy of the molecule. Intriguingly, we find that the treatment with a non-lytic variant indeed did not prolong graft survival in the MHC mismatch transplant model. While the lytic IL-15/Fc is not generally lymphoablative in mice—due to the fact that the IL-15R is only expressed on activated T cells—these results nonetheless suggest that complement and/or FcR mediated deletion of IL-15R expressing activated T cells and macrophages contributes to the overall immunoprotective effect. Interestingly, Smith et al. reported earlier that the use of a recombinant soluble IL-15R alpha subunit (sIL-15Rα) was ineffective in preventing graft rejection in the MHC mismatch heart transplant model, but did prolong graft survival in a minor histocompatibility mismatch mouse strain combination. An IL-15 neutralizing agent, such as sIL-15Rα, would not target IL-15R bearing cells for deletion by the innate immune system. We would therefore propose that while inhibition of the IL-15/IL-15R pathway is sufficient to prevent graft rejection and induce antigen-specific tolerance in a minor histocompatibility mismatch mouse heart transplant setting, Fc-mediated activation of the innate immune system and depletion of IL-15R bearing cells contributes to the prolonged graft survival of fully MHC mismatched heart transplants observed in this study.

In addition to prolonging graft survival, we find that a short course of treatment can induce antigen-specific tolerance in both, minor histocompatibility mismatched heart transplants, as well as in fully MHC-mismatched islet allografts. Furthermore, the fusion protein synergizes with the costimulation blocker antiCD 154 in preventing heart transplant rejection. A number of embodiments of the invention have been described. Nevertheless, it will be understood that various modifications may be made without departing from the spirit and scope of the invention. Accordingly, other embodiments are within the scope of the following claims.

What is claimed is:

1. A mutant interleukin-15 (IL-15) polypeptide comprising a naturally occurring IL-15 that has a deletion mutation of one or more of the first 48 amino acid residues of the signal sequence and a substitution mutation of one of the glutamine residues corresponding to the glutamine residues at positions 101 and 108 of SEQ ID NO:1.

2. The mutant IL-15 polypeptide of claim 1, comprising a substitution mutation of both of the glutamine residues corresponding to the glutamine residues at positions 101 and 108 of SEQ ID NO:1.

3. The mutant IL-15 polypeptide of claim 1, comprising the sequence of SEQ ID NO:2.

4. The mutant IL-15 polypeptide of claim 1, further comprising a leader sequence.

5. The mutant IL-15 polypeptide of claim 4, wherein the leader sequence comprises a CD5 leader sequence.

6. The mutant IL-15 polypeptide of claim 1, wherein the mutant IL-15 polypeptide is joined to a heterologous polypeptide that increases the circulating half-life of the mutant IL-15 polypeptide beyond that of the mutant IL-15 polypeptide alone.

7. The mutant IL-15 polypeptide of claim 6, wherein the heterologous polypeptide is the Fc region of an immunoglobulin.

8. The mutant IL-15 polypeptide of claim 6, wherein the Fc region is a mutant of a naturally occurring Fc region of an immunoglobulin.

9. The mutant IL-15 polypeptide of claim 8, wherein the naturally occurring Fc region is an Fc region of an immunoglobulin of the G class (IgG).

10. The mutant IL-15 polypeptide of claim 8, comprising the sequence of SEQ ID NO:7.

11. The mutant IL-15 polypeptide of claim 1, wherein the polypeptide is substantially free of heterologous biological agents.

12. A nucleic acid molecule encoding the mutant IL-15 polypeptide of claim 1.

13. A cell comprising the nucleic acid molecule of claim 12.

14. A pharmaceutically acceptable composition comprising a therapeutically effective amount of the mutant IL-15 polypeptide of claim 1.

15. A method of suppressing the immune response in a patient, the method comprising administering to the patient an amount of the mutant IL-15 polypeptide of claim 1 sufficient to inhibit a cellular event that normally occurs when wild-type IL-15 binds the IL-15 receptor complex in a cell of the patient.

16. A method of treating a patient who has been diagnosed as having, or who is predisposed to having, an autoimmune disease, the method comprising administering to the patient a therapeutically effective amount of the mutant IL-15 polypeptide of claim 1.

17. A method of treating a patient who has received, or who is scheduled to receive, a transplant of a biological tissue, the method comprising administering to the patient a therapeutically effective amount of the mutant IL-15 polypeptide of claim 1.

18. The method of claim 16, wherein the biological tissue comprises islet cells, cardiac myocytes, hepatocytes, osteocytes, neurons, or glial cells.

19. A dimer consisting of two identical polypeptides, the polypeptides comprising the mutant IL-15 polypeptides of claim 1.

20. A pharmaceutically acceptable composition comprising the dimer of claim 19.


SEQUENCE LISTING (30 sequences)

SEQ ID NO: 1 - protein, 114 aa, Homo sapiens
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 2 - protein, 114 aa, Artificial Sequence
  Synthetically generated peptide
NWVNVISDLKKIEDLIQSMHIDATLYTESDVHPSCKVTAMKCFLLELQVISLESGDASIHDTVENLIILANNSLSSNGNVTESGCKECEELEEKNIKEFLDSFVHIVDMFINTS

SEQ ID NO: 3 - protein, 24 aa, Homo sapiens
MPMGSLQPLATLYLLGMLVASCLG

SEQ ID NO: 4 - protein, 138 aa, Artificial Sequence
  Synthetically generated peptide
MPMGSLQPLATLYLLGMLVASCLGNWVNVISDLKKIEDLIQSMHIDATLYTESDVHPSCKVTAMKCFLLELQVISLESGDASIHDTVENLIILANNSLSSNGNVTESGCKECEELEEKNIKEFLDSFVHIVDMFINTS

SEQ ID NO: 5 - protein, 232 aa, Artificial Sequence
  Synthetically generated peptide
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 232 aa, Artificial Sequence
  Synthetically generated peptide
DPKSADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 346 aa, Artificial Sequence
  Synthetically generated peptide
NWVNVISDLKKIEDLIQSMHIDATLYTESDVHPSCKVTAMKCFLLELQVISLESGDASIHDTVENLIILANNSLSSNGNVTESGCKECEELEEKNIKEFLDSFVHIVDMFINTSDPKSADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 370 aa, Artificial Sequence
  Synthetically generated peptide
MPMGSLQPLATLYLLGMLVASCLGNWVNVISDLKKIEDLIQSMHIDATLYTESDVHPSCKVTAMKCFLLELQVISLESGDASIHDTVENLIILANNSLSSNGNVTESGCKECEELEEKNIKEFLDSFVHIVDMFINTSDPKSADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 17 aa, Artificial Sequence
  Synthetically generated peptide
MRYMILGLLALAAVCSA

SEQ ID NO: 10 - protein, 21 aa, Homo sapiens
MVLGTIDLCSCFSAGLPKTEA

SEQ ID NO: 11 - protein, 8 aa, Artificial Sequence
  Synthetically generated peptide
DYKDDDDK

SEQ ID NO: 12 - DNA, 21 bp, Artificial Sequence
  Primer
gcctggatgctaacagaagga

SEQ ID NO: 13 - DNA, 20 bp, Artificial Sequence
  Primer
gttcatcccgtcgctatggt

SEQ ID NO: 14 - DNA, 24 bp, Artificial Sequence
  Probe sequence
atgacaaggatgccgggcaagtgt

SEQ ID NO: 15 - DNA, 21 bp, Artificial Sequence
  Primer
aatctgtggctaccggtggta

SEQ ID NO: 16 - DNA, 24 bp, Artificial Sequence
  Primer
ggtggaagagctgatacattccta

SEQ ID NO: 17 - DNA, 25 bp, Artificial Sequence
  Probe sequence
atggttctggtggctctggttggaa

SEQ ID NO: 18 - DNA, 23 bp, Artificial Sequence
  Primer
gcaaagactggcttcatatccat

SEQ ID NO: 19 - DNA, 21 bp, Artificial Sequence
  Primer
gcagaagaggtgttccattgg

SEQ ID NO: 20 - DNA, 26 bp, Artificial Sequence
  Probe sequence
acaaggaccagctctgtccttggcag

SEQ ID NO: 21 - DNA, 20 bp, Artificial Sequence
  Primer
tgctcttcgggaaccaagct

SEQ ID NO: 22 - DNA, 19 bp, Artificial Sequence
  Primer
cagggttgctgggcagtga

SEQ ID NO: 23 - DNA, 28 bp, Artificial Sequence
  Probe sequence
caccagagcagttctcaacctggacagc

SEQ ID NO: 24 - DNA, 23 bp, Artificial Sequence
  Primer
acaatgaacgctacacactgcat

SEQ ID NO: 25 - DNA, 21 bp, Artificial Sequence
  Primer
tggcagtaacagccagaaaca

SEQ ID NO: 26 - DNA, 25 bp, Artificial Sequence
  Probe sequence
ttggctttgcagctcttcctcatgg

SEQ ID NO: 27 - protein, 6 aa, Homo sapiens
EPKSCD

SEQ ID NO: 28 - protein, 6 aa, Artificial Sequence
  Synthetically generated peptide
DPKSAD

SEQ ID NO: 29 - protein, 9 aa, Artificial Sequence
  Synthetically generated peptide
LLELQVISL

SEQ ID NO: 30 - protein, 5 aa, Artificial Sequence
  Synthetically generated peptide
ENLII